(19) United States
(12) Patent Application Publication
(10) Pub. No.: US 2023/0079100 A1
(43) Pub. Date: Mar. 16, 2023

(54) PROCESS FOR PREPARING SOLID FORMULATIONS COMPRISING A HYDROPHOBIC COMPOUND DISPERSIBLE IN A COLD LIQUID AND RELATIVE SOLID FORMULATIONS

(71) Applicant: NUTRILINEA S.R.L., Gallarate (VA) (IT)

(72) Inventors: Alessandro TADDEI, Gallarate (VA) (IT); Christian STOLLBERG, Gallarate (VA) (IT); Sebastiano Maurizio CASTORINA, Gallarate (VA) (IT); Arianna VANELLI, Gallarate (VA) (IT); Fabiana SABINO, Gallarate (VA) (IT)

(21) Appl. No.: 17/796,218

(22) PCT Filed: Sep. 20, 2021
(86) PCT No.: PCT/IB2021/058543

§ 371 Date: Jul. 28, 2022

(30) Foreign Application Priority Data
Sep. 18, 2020 (IT) 102020000022126

(51) Int. Cl. A61K 9/16; A61K 9/20; A61J 3/02

(52) U.S. Cl. CPC A61K 9/1623; A61K 9/1664; A61K 9/2013; A61K 9/1617; A61K 9/1658; A61K 9/1682; A61J 3/02; A61K 9/2018; A61K 9/2063; A61K 9/2068; A61K 9/2095

(57) ABSTRACT

The present invention relates to a process in a fluid-bed system for preparing solid formulations having high dispersibility in a cold liquid (room temperature 20° C.-25° C.±2° C.), preferably in water or in a water-based liquid, wherein said solid formulations comprise at least one hydrophobic compound. Furthermore, the present invention relates to said solid formulations obtained by means of said process and to the use thereof for the preparation of dietary supplements, foods for specialist medical purposes (in short FSMPs), pharmaceutical compositions, medical device compositions and/or cosmetic compositions.

The present invention relates to a process to be carried out in a fluid bed granulator system for preparing solid formulations having high properties of dispersion and/or solubilisation in a cold liquid (for example at room temperature comprised from 20° C.±2° C. to 25° C.±2° C.), preferably water or a water-based liquid, wherein said solid formulations comprise at least one hydrophobic compound. Furthermore, the present invention relates to said solid formulations (preferably in powdered form) with high dispersibility and/or solubilisation in a cold liquid (for example at room temperature comprised from 20° C.±2° C. a 25° C.±2° C.) obtained by means of said process and to the use thereof for the preparation of dietary supplements, foods for specialist medical purposes (in short FSMPs), pharmaceutical compositions, medical device compositions and/or cosmetic compositions.

In the field of dietary supplements, foods for special medical purposes (FSMPs), pharmaceutical and/or cosmetic compositions, the problem of having to process and/or formulate powders or solid compounds having low degree of dispersion and/or solubilisation in a cold liquid (room temperature), such as for example water or a water-based liquid, is frequently observed. The degree of dispersion and/or solubilisation of a powder, or solid particles in general, in water depends on the wettability thereof. When powders or solid particles (from 0.5 g to 3 g) with little or no wettability are added to the water (from 50 ml to 100 ml), or vice versa, they tend to form aggregates on the surface of the water and/or aggregates suspended in the water which are not able to disperse rapidly.

In the context of the present invention, the term “wettability” is used to indicate the process which brings a liquid and a solid surface into contact. The system is described by the contact angle (0), defined as the angle formed by the tangent to the liquid-fluid interface and by the tangent to the solid surface, at the contact line between the three phases. A low contact angle)(e<90° describes a situation in which the solid is partially wet by the liquid (hydrophilicity), while a high contact angle)(e>90° describes a situation in which the solid is scarcely wet (hydrophobicity). Wettability is a property that affects the obtainment both of superhydrophobic and superhydrophilic surfaces and it is evaluated by means of contact angle testing and measurement. Superhydrophilicity (strong affinity with water) confers maximum wettability to the surface, thanks to the contact angle between the drop and the surface smaller than about 5°, with the formation of a thin continuous film; on the contrary, in the presence of superhydrophobicity (low affinity with water), for contact angles greater than 150°, there is no wettability, facilitating the formation of isolated drops (FIG. 1).

In the context of the present invention, the term “powders” is used to indicate a very fine powder with X50<125 μm (for example from 50 μm to 125 μm), fine powder with X50 125-180 μm, moderately fine powder with X50 180-355 μm or coarse powder with X50>355 μm (for example from 355 μm to 1000 μm).

The use of hydrophobic compounds with health benefits which can be administered through the oral route, such as pharmaceutical or nutraceutical compositions or dietary supplements, may be limited by low or no wettability of said compounds in the aqueous environment of the digestive system. The low wettability inevitably leads to the aggregation of the solid particles. Said aggregates have significantly reduced bioavailability with respect to the individual particles which remain dispersed in aqueous environment. As a matter fact, hydrophobic compounds are known to be poorly absorbed by the gastrointestinal tract (GI). Therefore, in order to achieve the therapeutic effect by hydrophobic compounds there often arises the need to increase the dosage so as to have the absorption of the desired amount of compound.

Furthermore, a rapid and homogeneous dispersion and/or dissolution of solid formulations comprising hydrophobic compounds in a cold liquid is fundamental to ensure the correct intake of the predetermined dose by the consumer subject and for the compliance of the consumer subject with the intake of the composition. For example, when a powder not suitably dispersed in a liquid it remains attached to the container (for example, a glass or plastic cup) and/or to the lips of the consumer, the full intake of the predetermined dosage is not guaranteed. Furthermore, the intake may not be comfortable for the consumer due to the formation of aggregates on the surface of the liquid, particularly during swallowing, possibly causing coughing. Besides being bothersome, said cough can cause the ejection of the solid composition dispersed in the liquid administered to the subject, with ensuing reduction of the dose taken. Failure to take the entire expected dose of said solid composition, due to the various aforementioned reasons, may lead to a reduced or no therapeutic effect by the administered solid composition.

There are various processes used in the pharmaceutical, nutraceutical and/or cosmetic industry to render the solid compositions comprising hydrophobic compounds that are easily dispersible in water, such as for example the dry granulation or mixing of hydrophobic powders with charging agents, or granulation with charging agents under wet conditions.

Said processes known in the art do not always render the powders sufficiently dispersible and/or dissolvable in water, or as processes they are inefficient in terms of processing time and/or in terms of cost-effectiveness.

For example, document US 2012/238516 discloses a method for preparing anthelmintic compositions comprising two or more anthelmintic agents. Said compositions are in the form of granules dispersible in water. Mixtures of wetting agents dissolved in one or more solvents are atomised in the preparation of these compositions, for example Tween 80 and benzyl alcohol can be used. However, this process is often not effective in increasing the wettability of hydrophobic active agents, and provides for longer processing times to allow the evaporation of the solvent.

Document US 2001/006650 describes a process for the preparation of capsules comprising (i) a hydrophobic long chain fatty acid; (ii) a surfactant; and (iii) a therapeutic agent which—in mixture—form a solid solution at room temperature. However, mixtures of wetting agents different from each other are used in this document. Furthermore, the wetting agents used have a very high hydrophobic portion which makes the dispersion thereof in water difficult. For this reason, the wetting agents are in turn inserted into tablets for final use.

In the light of the above, it is clear that there arises the need to identify novel preparation processes which allow to optimise the dispersion of hydrophobic active agents in water, thus allowing to increase the availability thereof and ease of use by the end consumer.

Following an extensive research and development phase, the Applicant addresses and solves the aforementioned technical problems by providing a process in a fluid bed granulator system for preparing a solid formulation that is highly and readily dispersible in a cold liquid (for example at room temperature comprised from 20° C.±2° C. to 25° C.±2° C.)) comprising at least one hydrophobic compound, wherein said process comprises the step of depositing a wetting agent (preferably polysorbate) on the surface of a solid substrate comprising said at least one hydrophobic compound, by atomising the wetting agent on said moving solid substrate by means of a process air in the fluid bed (in short, process of the invention).

By means of said process of the invention, a solid formulation is prepared that is highly and readily dispersible in a cold liquid comprising a hydrophobic compound (in short, solid formulation of the invention) that can be used for the preparation of solid compositions to be administered to subjects in need, such as dietary supplements, foods for special medical purposes (FSMPs), pharmaceutical compositions, compositions for medical devices (Regulation EU 2017/745 (MDR)) and/or cosmetic compositions (in short, compositions of the invention).

The hydrophobic compounds comprised in the solid formulations or solid compositions of the present invention have a high gastrointestinal absorption and, therefore, high bioavailability following an oral administration. Furthermore, said solid formulations and compositions of the present invention can be easily administered due to the high and rapid dispersion and/or dissolution thereof in a cold liquid (room temperature), such as water or water-based beverage, which guarantees the administration of the entire dosage of the hydrophobic compounds. Lastly, said solid formulations of the invention can be used in mouth dissolvable compounds, thanks to their high degree of wettability in water.

As a result, the present invention solves the problem of the formation of a powder coating on the surface of a liquid once a solid formulation or composition (e.g. powder or granules) is mixed with a liquid (e.g. water), as well as the problem of the formation of cakings in the packages of said solid formulations or compositions (e.g. stick-pack or sachet).

Furthermore, the solid formulations and the solid compositions thereof of the present invention show good palatability for the consumer.

Lastly, the process of the present invention for the preparation of solid formulations dispersible in a cold liquid comprising at least one hydrophobic compound is efficient in terms of processing time, type of process machines, and it is cost-effective (low energy consumption costs).

As known, in the pharmaceutical and food industry it is preferable to have compounds and compositions in solid form given that they are easier to handle, process and store.

These and other objects which will be apparent from the detailed description that follows, are achieved by the solid formulations and by the solid compositions of the present invention thanks to the technical characteristics detailed in the description and in the attached claims.

### FIGURES

FIG. 1 shows the theory of the wettability of a solid with a liquid.

FIG. 2 shows a diagram of the process according to the present invention.

FIG. 3A shows a test of dispersion of a solid substrate in water (comprising a hydrophobic compound) before being subjected to the process according to the invention.

FIG. 3B shows a test of dispersion of a solid formulation in water prepared according to the invention (solid substrate subjected to the process according to the invention).

FIG. 4 schematically shows a process chamber of a fluid bed system, an atomisation means, a wetting agent exiting from said atomisation means, and a solid substrate (powder) in vertical motion by means of the process air.

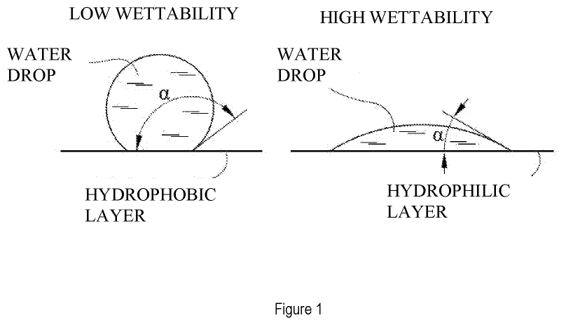

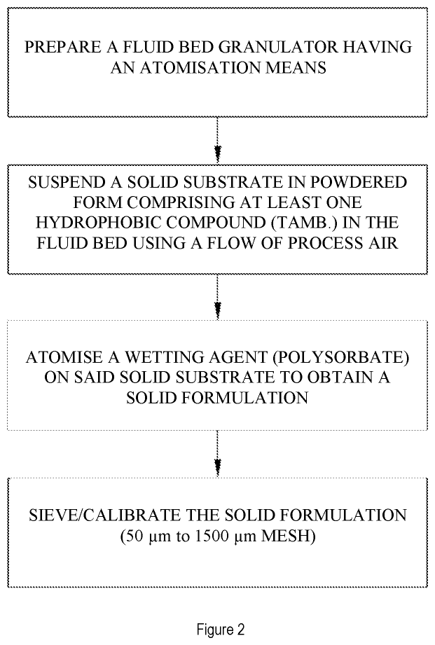

### DETAILED DESCRIPTION OF THE INVENTION

The process according to the present invention for the preparation of a solid formulation that is highly and readily dispersible in a cold liquid (room temperature), comprising at least one hydrophobic compound and a wetting agent, provides for the use of a fluid-bed granulator system (or reactor) (in short, system of the invention or fluid bed system).

A fluid bed system is characterised by a process air distribution system, a process chamber and means for atomising a wetting agent in liquid form. Said process air moves (by driving)—inside the process chamber—a solid substrate in powdered form comprising at least one hydrophobic compound to be treated with the wetting agent. The displacement of the solid substrate in the form of powder ensures the separation of the particles forming said solid substrate, so that the surfaces of said particles are separated from each other and exposed to the wetting agent as much as possible during the atomisation process. This allows the solid substrate particles to be reached by the wetting agent particles (preferably a polysorbate) atomised by means of the atomiser.

The process of the present invention comprises or, alternatively, consists the steps of:

(I) providing a fluid bed granulator system comprising a process chamber, a process air distribution system, means for atomising a wetting agent in liquid form (as defined in the context of the present invention), preferably wherein said atomising means is a nozzle atomiser, and providing a peristaltic pump (integrated or not integrated in the fluid bed system), wherein said atomising means is positioned so as to be able to atomise the wetting agent in the process chamber);

(II) parallel or subsequently (before or after) to step (I), providing a wetting agent in liquid form, preferably an emulsifier such as, for example, a polysorbate (e.g. a liquid polysorbate with purity of about 100% w/w, without dilution in water), in a wetting agent container (integrated or not integrated in the fluid bed system), preferably at room temperature, and connecting said wetting substance container by means of connection means (for example pipes, such as pipes made of plastic material) to said peristaltic pump;

(III) transferring (for example suctioning)—by means of a first transfer means (for example a lance)—a solid substrate in powdered form comprising at least one hydrophobic compound (as defined in the context of the present invention) from a solid substrate container (integrated or not integrated in the fluid bed system) to said process chamber of the fluid bed system;

(VI) suspending said solid substrate in said process chamber in a flow of said process air by imparting a drive motion (for example, elliptical motion or rectilinear motion) to said solid substrate, wherein the operating conditions of the fluid bed system are preferably the following:
cubic meters per hour of said process air from 30 m³/h to 2700 m³/h, preferably from 80 m³/h to 2500 m³/h, more preferably from 110 m³/h to 2300 m³/h,
temperature of said process air from 15° C. to 30° C., preferably from 18° C. to 28° C., more preferably about 25° C.;

(V) atomising—by means of said atomisation means—said wetting agent in the process chamber to obtain a solid formulation of step (V) comprising said solid substrate and said wetting agent, wherein said solid substrate comprises or, alternatively, consists of at least one hydrophobic compound;

(VI) optionally, sieving or calibrating (for example by means of certified sieves or laser particle size analyser) the solid formulation obtained from step (V) to obtain a solid formulation of step (VI) having an average particle size comprised from 50 μm to 1500 μm, preferably from 80 μm to 1200 μm, more preferably from 100 μm to 1000 μm; and

(VII) optionally, transferring—by means of a second transfer means (for example, a lance)—said solid formulation of step (V) or step (VI) from the process chamber to a solid formulation container (preferably outside the fluid bed system).

In a preferred embodiment of said fluid bed system, the process air imparts—to powdered solid substrate present in the process chamber—an elliptical or continuous circular crown motion (or movement), wherein said solid substrate is channelled and directed at a predetermined angle (for example from 90° C. to 100° C.). As a result, the individual particles of the powders are separated so as to prevent the particles from being contiguous or close to each other and so as to expose the surface of each individual particle as much as possible so as to be impacted by the atomised wetting agent (preferably a polysorbate). The elliptical or continuous circular crown movement allows the frequent passage of the individual powder particles in the areas for atomising the wetting agent (preferably a polysorbate) by the atomisation means, so as to obtain the predetermined amount of wetting agent in a short time, for example from 10 minutes to 120 minutes, preferably from 20 minutes to 100 minutes, more preferably from 25 minutes to 80 minutes. Alternatively, the fluid bed system may be structured so that hat said process air imparts—to the powdered solid substrate present in the process chamber—an approximately rectilinear motion (vertical or horizontal with respect to the main axis of the process chamber) (FIG. 4). Generally, this rectilinear motion of the solid substrate leads to a number of smaller passages in the time unit of the individual powder particles in the areas for atomising the wetting agent with respect to the elliptical motion described above. Therefore, in order to deposit the same amount of wetting agent per solid substrate unit, in a fluid bed system with rectilinear motion there is required a longer time with respect to a fluid bed system with elliptical motion, considering the other conditions of the system to be equal.

In order to optimise the movement of the individual powder particles of the solid substrate in the process of the invention (according to any one of the embodiments), the fluid bed system is advantageously loaded with said solid substrate in powdered form in a percentage by weight comprised in a range from 20% to 70%, preferably from 30% to 60%, more preferably from 40% to 50%, with respect to the total volume of the process chamber.

In the atomising step (V) the wetting agent is adsorbed on the solid substrate spreading on part or on all of the surface of the particles of the solid substrate. For example, the wetting agent binds to the solid substrate through the intermolecular and van der Waals forces and spreads inhomogeneously (or partially homogeneously) on the surface of the particles of the solid substrate (spot-coating).

The spraying step (V) is carried out by actuating said peristaltic pump which supplies the atomisation means with the wetting agent and generates an atomising compressed air which drives and it atomises the wetting agent. The pressure of said atomising compressed air that exits from the atomisation means is preferably comprised in the range of from 0.1 bar to 10 bar, preferably from 0.2 bar to 6 bar, more preferably from 0.3 bar to 4 bar (for example 1 bar, 2 bar or 3 bar) (1 bar=10⁵ Pa). A power gradient is preferably applied to the peristaltic pump depending on the type amount of wetting substance to be atomised per minute, for example a power gradient from 10% up to 100% in a time interval from 30 minutes to 100 minutes. The amount of wetting agent (preferably a polysorbate) applied in the time unit can be monitored by means of a flowmeter present in the fluid bed system or by means of a weighing scale on which the wetting substance container is arranged.

In a preferred embodiment, the atomising step (V) is carried out in a manner such to obtain a solid formulation comprising said wetting agent (preferably a polysorbate) in a percentage by weight comprised from 0.5% to 30% with respect to the weight of the solid formulation, preferably from 1% to 20%, more preferably from 2% to 10% (for example, 3%, 4%, 5%, 6%, 7%, 8%, 9%, 11%, 12%, 13%, 14% and 15%).

The atomisation means is preferably a nozzle atomiser which atomises the wetting agent in liquid form (preferably a polysorbate) with two or more slits present on the nozzle (slit with a varible width for example from 0.15 mm to 0.30 mm, preferably from 0.2 mm to 0.25 mm), advantageously at different compressed air pressures to obtain the finest drops of wetting agent possible, preferably in a range of a diameter X50 from about 0.2 μm to 1 μm.

The at least one hydrophobic compound (a hydrophobic compound or a mixture of hydrophobic compounds) comprised in the solid substrate in powdered form used in the process of the present invention (and, therefore, comprised in the solid formulation of the present invention) may be selected from the group comprising or, alternatively, consisting of: plant extracts, vitamins, minerals, amino acids, plant and animal solid fats, whey and casein proteins, polyphenols and mixtures thereof, or any other hydrophobic compound suitable to be subjected to the process of the invention.

The wetting agent used in the process of the present invention (and, therefore, comprised in the solid formulation of the present invention) may be selected from an emulsifier, preferably in the group comprising or, alternatively, consisting of: a polysorbate (for example, polysorbate 20, polysorbate 40, polysorbate 60 and polysorbate 80), a sucrester (a sucrose fatty acid ester), or mixtures thereof.

In a preferred embodiment of the present invention (process and solid formulation) the wetting agent comprises or, alternatively, consists of a polysorbate. Advantageously, said wetting agent comprises or, alternatively, consists of a polysorbate not comprising water (for example a liquid polysorbate with purity from 80% to about 100%, such as 85%, 90%, 95%, 96%, 97%, 98%, 99%, or 99.5%).

In one embodiment of the present invention the wetting agent is, consists of a polysorbate not mixed with other wetting agents or solvents.

In the context of the present invention, the expression “pure polysorbate” is used to indicate a polysorbate which does not contain water or other solvents, traces at most.

Polysorbates are a class of emulsifiers for pharmacological, cosmetic and food use. They are copolymers obtained by the combination of sorbitol and ethylene oxide esterified with fatty acids. Polysorbates are obtained from sorbitan esters by etherifying free hydroxyl groups with ethylene oxide. About 20 moles of ethylene oxide are fixed for each mole of ester. Hydrophilic properties are increased by introducing oxyethylene groups. The most common polysorbates include: polysorbate 20 (polyoxyethylensorbitane monolaurate) comprised among additives E432 (European classification); polysorbate 40 (polyoxyethylensorbitane monopalmitate); polysorbate 60 (polyoxyethylensorbitane monostearate); and polysorbate 80 (polyoxyethylene sorbitan monooleate) comprised among additives E433 (European classification) and commercially known for example as Tween® 80.

Surprisingly, polysorbates have proven to function very effectively as wetting agents once applied on solid substrates in powdered form having low or no wettability, in order to achieve the objects of the present invention.

For example, in the process of the present invention there can be used respectively as a hydrophobic compound and as wetting agent: a wetting agent: a plant extract (e.g. milk thistle (Silybum Marianum)) and a polysorbate; boswellia (e.g. boswellia serrata titrated to 65% in boswellic acids) and a polysorbate; alpha-lactalbumin and a polysorbate; or palmitoylethanolamide (PEA) and a polysorbate.

Furthermore, the process according to the present invention is effective in increasing the dispersibility of mixtures of amino acids, proteins (e.g. whey protein isolate 90%), hemp oil, and fructo-oligosaccharides. Furthermore, sequential application of hemp oil (liquid) and polysorbate 80 (liquid) on a soluble fibre such as FOS (solid), which allows to obtain a solid powder formulated from these three components (FOS, hemp oil, polysorbate) is particularly advantageous. This allows—with a single process—to obtain a powder soluble in water containing an oil (hemp oil in this case), which—once dissolved in water—does not have any oil patches on the surface thanks to polysorbate 80, which dispersed/emulsified the oil.

Furthermore, the process according to the present invention allows to increase the dispersibility of the coacervates in water. Coacervates are systems which allow the microencapsulation of active substances through coacervation in aqueous and organic solvents. However, the polymers used to obtain the microcapsules through coacervation are hydrophobic and they tend to float once dispersed in water. Therefore, the process according to the present invention may advantageously be used to increase the wettability of microcapsules obtained through coacervation. In particular, the Applicant surprisingly found that atomising a pure polysorbate according to the process of the present invention allows to obtain microcapsules obtained through coacervation which can be advantageously dispersed in water. Preferably, the final concentration of polysorbate may be comprised from 6%-10% with respect to the total weight of the coacervate+polysorbate mixture.

The highly and readily wettable solid formulation, preferably in powdered form, obtained from the process of the present invention (in short solid formulation of the invention) comprises or, alternatively, consists of: (i) a solid substrate comprising or, alternatively, consisting of at least one hydrophobic compound, and (ii) a wetting agent, preferably a polysorbate, as described in the present invention.

The solid formulation of the invention preferably comprises said wetting agent in a percentage by weight comprised from 0.5% to 30% with respect to the weight of the solid formulation, preferably from 1% to 20%, more preferably from 1% to 10% or from 5% to 10% (for example, 2%, 3%, 4%, 6%, 7%, 8%, 9%, 11%, 12%, 13%, 14% and 15%).

Advantageously, the solid formulation of the invention comprises or, alternatively, consists of: (i) the solid substrate (comprising or, alternatively, consisting of at least one hydrophobic compound) from 70% to 99.5% and (ii) the wetting agent from 0.5% to 30%, wherein said percentages are percentages by weight with respect to the weight of the solid formulation; preferably (i) from 80% to 99% and (ii) from 1% to 20%, more preferably (i) from 90% to 98% (e.g. 93%, 95%, 97%) and (ii) from 2% to 10% (e.g. 7%, 5%, 3%).

According to a preferred aspect, the wetting agent is a polysorbate (for example polysorbate 20 or 40 or 60 or 80) and the solid formulation comprises said polysorbate in a percentage by weight from 1% to 10% or from 5% to 10% (for example, 2%, 3%, 4%, 5%, 6%, 7%, 8%, 9%, with respect to the weight of the solid formulation.

The highly and readily wettable solid formulation obtained by the process of the present invention (according to any one of the described embodiments or aspects) preferably has an average particle size value X50 (or average diameter of the solid particles of the solid formulation of the invention) comprised from 50 μm to 1500 μm, preferably from 80 μm to 1200 μm, more preferably from 100 μm to 1000 μm. Said solid or powder particle size values are determined by means of instruments and methods known to the person skilled in the art (for example, certified sieves, or laser particle size analysers).

The highly and readily wettable solid formulation obtained by the process of the present invention may be mixed with further active ingredients (preferably hydrophilic) and/or additives and/or excipients to obtain compositions (in short, composition of the invention), such as dietary supplements, foods for special medical purposes (in short, FSMPs), pharmaceutical compositions and/or compositions for medical devices.

Said compositions of the invention, comprising the solid formulations obtained by the process of the invention, are preferably in solid form, such as powder, granules, capsules or tablets, for example and packaged in blisters, stick-packs or sachet.

Alternatively, said compositions of the invention may be in liquid form, wherein the solid formulations obtained by the process of the invention are dispersed in a liquid (for example, water or a water-based liquid, such as a beverage).

From the experimental tests carried out and reported below, it was observed that by means of the process of the present invention there are obtained solid formulations comprising at least one hydrophobic compound wherein said solid formulations have excellent wettability in a cold liquid (water or aqueous liquid) (room temperature) using a relatively low percentage by weight of wetting agent (preferably a polysorbate), as defined in the present invention.

Furthermore, it has surprisingly been observed that if a polysorbate is used diluted in water at low concentrations (for example, at a concentration of 10 grams of polysorbate on 90 g of water), it is not able to improve the dispersion—in a cold liquid—of the tested powdered solid substrates, with a poor or insufficient degree of dispersion (measured visually). On the contrary, if polysorbate is used at a high degree of purity (for example, concentration of about 100% w/w), the results show an effective improvement of the dispersion of the tested powdered solid substrates in a cold liquid, with an excellent degree of dispersion (measured visually).

It is therefore clear that it is particularly advantageous to use a wetting agent consisting of at least one polysorbate not diluted in water or in other solvents, or even less mixed with wetting agents other than polysorbates. In a preferred embodiment, the wetting agent consists of a pure polysorbate 80, i.e. not diluted/mixed in water or in other solvents.

Lastly, the process of the invention is efficient, having a short processing time and low energy consumption costs. In the case of use as a wetting agent of a polysorbate not diluted in water (for example, a polysorbate with purity of about 100% w/w), the high efficiency of said production parameters (processing time and energy consumption) is mainly, but not exclusively, due to the fact that the process is carried out at room temperature, since a step for evaporating the water is not necessary, given that said wetting agent is devoid of water.

If the solid formulation of the invention is dispersible in a liquid (or it has improved dispersion/dissolution properties), it can be determined by the average particle size obtained when the solid formulation produced using the process of the invention is mixed with water. The particle size can be measured using known methods. In the context of the present invention, it is defined that the solid formulation of the invention is not dispersible in a liquid (for example water) if it mainly forms aggregates or cakings with average-size particles (average diameter) greater than about 200 μm. According to an aspect of the present invention, the solid formulation of the invention, when mixed with water, produces a particle population in which from 50% to 100% of said particle population has an average diameter comprised from 1 μm to 100 μm, preferably from 1 μm to 50 μm. For example, from 50% to 80% of the particle population may have an average diameter comprised from 1 μm to 100 μm, or at least 60% of the particle population may have an average diameter comprised from 1 μm to 50 μm, or at least 70% of the particle population may have an average diameter comprised from 1 μm to 50 μm, or at least 50% of the particle population may have an average diameter comprised from 1 μm to 40 μm, or at least 50% of the particle population may have an average diameter comprised from 1 μm to 30 μm.

In the context of the present invention, the expression “in a cold liquid” in the phrase “solid formulations having high properties for dispersion and/or solubilisation in a cold liquid” or the like, is used to indicate that the solid has properties for dispersing and/or solubilising in a liquid in the absence of a process for heating said liquid, i.e. “in a liquid at room temperature”.

In the context of the present invention, the expression “room temperature” is used to indicate a temperature (T) comprised in a range from 15° C. to 35° C., preferably from 20° C. to 30° C., more preferably from 20° C. to 25° C.±2° C., or ±1° C. as a function of the precision of the measuring instrument.

In the context of the present invention, the term “at least one pharmaceutical, food or cosmetic grade additive and/or excipient”, is used to indicate a substance devoid of therapeutic activity suitable for pharmaceutical, food or cosmetic use. In the context of the present invention, the additives and/or excipients acceptable for pharmaceutical, food or cosmetic use comprise all the ancillary substances known to the person skilled in the art for the preparation of compositions in solid, semi-solid or liquid form, such as, for example, diluents, solvents (including water, glycerine, ethyl alcohol), solubilisers, acidifiers, thickeners, sweeteners, flavour enhancement agents, colorants, lubricants, surfactants, preservatives, pH stabilizing buffers and mixtures thereof.

Unless specified otherwise, the expression composition or mixture or other comprising a component at an amount “comprised in a range from x to y” is used to indicate that said component can be present in the composition or other at all the amounts present in said range, even though not specified, extremes of the range comprised.

Experimental Part

1. Process According to the Invention

Dry Granulations in a Fluid Bed Granulator in the Absence of Charging Agents (According to the Invention)

Dry granulations were carried out in a fluid-bed granulator with nozzle atomiser, according to the invention, in the absence of charging agents, on a scale of size from 1 Kg to 2.5 Kg.

1.1. Materials

Solid substrates in powdered form:
Palmitoylethanolamide PEA (<50 μm): purity 99.7% (w/w), palmitoyldiethanolamide PDEA) absent, water content 0.2% (w/w), melting point 99.7° C.;
alpha-lactalbumin: obtained from sweet whey using ion exchange technology, composition (% w/w) proteins (on dry basis) 97.3±0.4 (Leco Combustion AOAC990.03), alpha-lactalbumin (% protein) 92.5±2.5 (HPLC-USP NF), pH 6.5±0.3 (10% SOL@20° CAOAC945.27);
Boswellia (Boswellia serrata, titrated to 65% in boswellic acids): obtained by extraction of boswellia serrata resin gum in ethyl acetate, identification: TLC; density (gm/mL): tap density 0.45-0.75, bulk density 0.20-0.50 (USP 37<616>); particle size range: 100% through 30# (USP 37<786>); and
Milk thistle (Silybum Marianum): obtained by extracting the seeds with water and ethanol; characterisation: composition: Silybum Marianum 80%, maltodextrin, 0.5% anhydrous silica; loss on drying 0.5%; density (tap density)˜0.5 g/mL.
Carob flour (PF1) (Ceratonia siliqua (Carob)): brown powder obtained from carob (seeds and/or pulp of the pod). The carob flour used in the present context is of food grade and it may be purchased commercially. For example, it may be purchased from Biosearch life s.a.

Wetting Agents:

a range of emulsifiers were tested, including polysorbates (polysorbate 80, polysorbate 60, polysorbate 40, polysorbate 20) and sucresters), obtaining similar results. By way of example and representation reported below are the tests carried out with:
liquid polysorbate 80, purity about 100% w/w (Tables I-III).

The polysorbate 80 (polyoxyethylene sorbitan monooleate, E433) used in the present experimental test is the commercial product Montanox Polis 80 obtained by reacting sorbitol with fatty acids (derived from olives) to obtain sorbitan esters which are reacted with ethylene oxide to obtain unprocessed polysorbate 80 which is subsequently subjected to decolourisation, neutralisation and filtration to obtain the desired product.

1.2. System

The fluid bed system used for the experimental tests is characterised by a process chamber, a process air distribution system, a nozzle atomiser with 2 different atomisation pressures.

Polysorbate 80 (pure at 100%, in liquid form without dilution with water) was atomised and applied in a percentage by weight of about 10%, 7% or 5% with respect to the weight of the solid formulation consisting of (a) solid substrate in powdered form consisting of a hydrophobic compound and (b) a wetting agent (Tables-Ill). The amount applied (10%, 7% and 5% w/w) proved to be sufficient to confer an excellent wettability to the hydrophobic compounds in the form of treated powders.

Conditions of the system used:
process air for suspending the powdered solid substrate: from 150 m³/h to 2700 m³/h;
temperature of said process air 25° C.-35° C.;
pressure of the atomising compressed air exiting from the atomisation means 0.5 bar to 2 bar (or 0.5·10⁵ Pa to 2·10⁵ Pa).

1.3. Visual evaluation of the degree of wettability of the solid formulation obtained by the process of the invention.

The solid formulations (1.5 g), comprising a solid substrate and a wetting agent according to Tables I-Ill (obtained by the process of the invention), were poured into 100 ml of water at room temperature (about 20° C.-25° C.±2° C.).

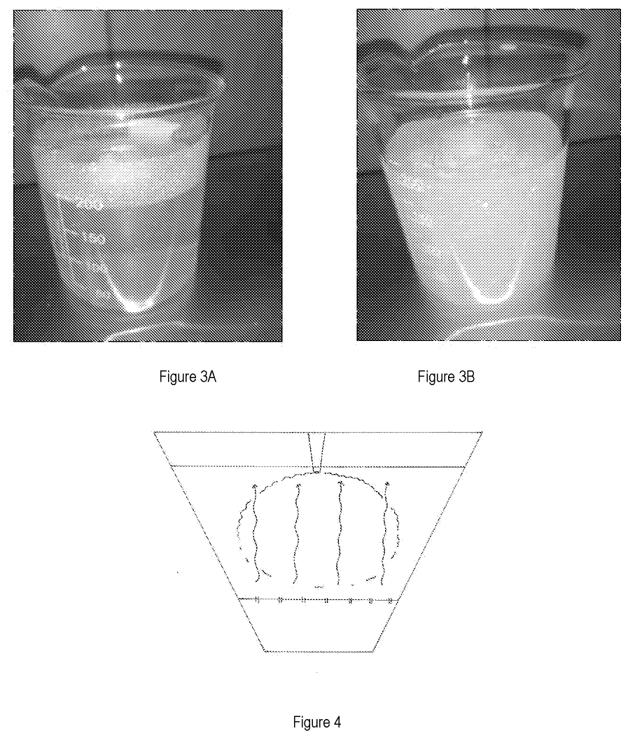

Initially no stirring is carried out, but a partial dispersion of the powdered solid formulation is visually observed. Stirring is subsequently carried out with a magnetic stirring bar for about 1 minute and it is visually observed that the powder is completely dispersed in the form of a suspension (FIG. 3B). After about 5 minutes from the end of the stirring, the powder is fully dissolved.

FIG. 3A represents the absence of dispersion of a solid substrate not subjected to the process of the invention with a wetting agent in water, and the formation of residues or aggregates on the water surface.

TABLE I
Application of the liquid polysorbate 80 by
means of a fluid bed system

| | | Visual degree of |
|---|---|---|
| Solid substrate | Wetting agent | wettability of the |
| (90% w/w, 900 g) | (10% w/w, 100 g) | solid formulation |
| PEA | Liquid polysorbate | Excellent |
| Alpha-lactalbumin | 80 pure at 100% | Excellent |
| Boswellia | | Excellent |
| Milk thistle | | Excellent |

TABLE II
Application of the liquid polysorbate 80 by
means of fluid bed system

| | | Visual degree of |
|---|---|---|
| Solid substrate | Wetting agent | wettability of the |
| (93% w/w, 930 g) | (7% w/w, 70 g) | solid formulation |
| PEA | Liquid polysorbate | Fair |
| Alpha-lactalbumin | 80 pure at 100% | Fair |
| Boswellia | | Fair |
| Milk thistle | | Fair |

TABLE III
Application of the liquid polysorbate 80 by
means of fluid bed system

| | | Visual degree of |
|---|---|---|
| Solid substrate | Wetting agent | wettability of the |
| (95% w/w, 950 g) | (5% w/w, 50 g) | solid formulation |
| PEA | Liquid polysorbate | Fair |
| Alpha-lactalbumin | 80 pure at 100% | Fair |
| Boswellia | | Fair |
| Milk thistle | | Fair |

1.4 Contact angle measurements on hydrophobic substrates treated according to the process of the present invention (see section 1 of the experimental part).

The experimental measurements reported hereinafter relate to the study of the wettability characteristics of powdered samples before and after the process according to the present invention. The determination of the static contact angle of ultrapure water (also referred to as grade 1) was carried out on the surface of the sample pelleted or deposited in the form of film, according to methods known in the state of the art and as reported in [1] Nowak et al. Powder Technology 233 (2013) 52-64, e in [2] Bachmann and McHale, European Journal of Soil Science, 60 (2009) 420-430. The determinations were carried out using a Krüss Easy Drop instrument using drops of 8 μL of milliQ water. The behaviour of the drop in the first seconds from the deposition was analysed by recording 30-second video at 25 fps (frames per second): the contact angle considered is the equilibrium value or the final one at the end of the recording. Each sample was analysed both deposited in the form of film and pelleted and at least 3 independent contact angle determinations were carried out for each sample.

5 different types of powders were analysed; each of them was analysed both in the unmodified form thereof (as such) and after the process according to the present invention. In the case of the PEA (Palmitoylethanolamide) powder, a second batch of sample was analysed, for which only measurements on film were carried out. Overall, determinations were carried out on a total of 12 samples as reported in Table IV. The results of the determinations are reported in Table IV: the mean contact angle value with standard deviation relative to the measurements on pellet and on film are reported. In case of complete wettability of the sample, an angle of 0° is reported. Overall, a greater correspondence of the wettability data measured on film with the dispersibility data observed experimentally (visual observation) is observed. This observation is in good agreement with the literature [1]; the less representativeness of the measurements on pellets can be linked in some cases to absorption phenomena linked to the porosity of the pellet or to alterations of the surface free energy of the powder during pelletisation due to the applied pressure [1].

TABLE IV

| | | Contact angle | Contact angle |
|---|---|---|---|
| N° | Samples | on pellet | on film (°) |
| 1 | PEA as such | 72 ± 4 | 117 ± 7 |
| 2 | PEA* sucrose fatty acid | 74 ± 2 | 117 ± 16 |
| | esters | | |
| 3 | PEA as such second | N.D. | 114 ± 5 |
| | batch | | |
| 4 | PEA+ polysorbate as | N.D. | 0 |
| | defined in section 1.1 | | |
| | (materials) | | |
| 5 | PF1 (carob flour) as | 0 | 114 ± 17 |
| | such | | |
| 6 | PF1+ polysorbate as | 0 | 0 |
| | defined in section 1.1 | | |
| | (materials) | | |
| 7 | milk thistle (silymarin) | 50 ± 5 | 73 ± 1 |
| | as such | | |
| 8 | Milk thistle | 0 | 56 ± 13 |
| | (silymarin) + polysorbate | | |
| | as defined in section 1.1 | | |
| | (materials) | | |
| 9 | Boswellia serrata as | 120 ± 18 | 137 ± 1 |
| | such | | |
| 10 | Boswellia serrata* | 0 | 0 |
| | sucrose fatty acid esters | | |
| 11 | Alpha-lactalbumin as | 61 ± 6 | 69 ± 3 |
| | such | | |
| 12 | Alpha-lactalbumin + | 52 ± 12 | 26 ± 10 |
| | polysorbate as defined | | |
| | in section 1.1 | | |
| | (materials) | | |

From the contact angle measurements reported above, it is concluded that the process according to the present invention allows to significantly increase the wettability of the materials tested. These measurements confirm what was observed at macroscopic level in the dispersibility tests carried out before and after treatment (section 1.3).

1.b Process According to the Invention

The process according to the present invention, as described in section 1 reported above, is also used to process the following active materials:
Mixtures of amino acids as reported in tables (a-d). Mixtures of amino acids are prepared using commercially available amino acids, purchased for example from Cambridge Commodities ltd. The AAs purchased are L-phenylalanine, L-lysine hydrochloride, L-methionine, L-cysteine, L-isoleucine, L-leucine, L-valine, L-histidine HCl, L-tyrosine, L-threonine, and L-tryptophan, all of which are food grade.
Protein (Whey protein isolate 90%). The protein used was purchased from Volac and it is a whey protein isolate 90%; agglomerated with sunflower lecithin.
Food hemp oil purchased from A.C.E.F, a chemical and pharmaceutical company. It has a pale yellow/transparent appearance and it contains palmitic acid (4%-9%), stearic acid (1%-4%), oleic acid (6%-18%), linoleic acid (46%-65%), alpha linoleic acid (14%-28%) and it has a food grade purity.
FOS (fructo-oligosaccharides). The FOS used were purchased from Beghin Meiji, under the trade name Actilight® 950P. They are a short-chain fruit-oligosaccharide powder (degree of polymerisation comprised from 3 to 5) obtained from sucrose.

TABLE a

| Mixture A: Ingredient (Tit. + possible surd.) | % weight |
|---|---|
| LEUCINE CAMBRIDGE 80 MESH tit. 100% IAF | 26.295 |
| ISOLEUCINE CAMBRIDGE 80MESH tit. 100% IAF | 13.148 |
| VALINE CAMBRIDGE 40MESH tit 100% P2202 IAF | 13.148 |
| L-LYSINE HCL tit. 100% IAF P1210 | 18.078 |
| L-PHENYLALANINE tit. 100% IAF P1608 | 1.972 |
| L-THREONINE tit. 100% IAF P2002 | 7.231 |
| L-METHIONINE tit. 100% IAF P1317 | 1.315 |
| Histidine HCL MONO CAMBRIDGE 80 MESH tit. 74% IAF | 3.554 |
| CYSTEINE BASE CAMBRIDGE 80 MESH tit. 100% IAF | 3.944 |
| L-TRYPTOPHAN tit. 100% IAF P2008 | 0.657 |
| TYROSINE CAMBRIDGE 80 MESH tit. 100% IAF | 0.657 |
| POLYSORBATE MONTANOX 80 LIQUID FOOD | 10 |
| TOTAL | 100 |

TABLE b

| Mixture B: Ingredient (Tit. + possible surd.) | % weight |
|---|---|
| LEUCINE CAMBRIDGE 80 MESH tit. 100% | 45 |
| ISOLEUCINE CAMBRIDGE 80 MESH tit. 100% | 22.5 |
| VALINE CAMBRIDGE 40 MESH tit. 100% | 22.5 |
| POLYSORBATE MONTANOX 80 LIQUID FOOD | 10 |
| TOTAL | 100 |

TABLE c

| | % |
|---|---|
| Mixture C: Ingredient (Tit. + possible surd.) | weight |
| LEUCINE CAMBRIDGE 80 MESH tit. 100% | 60 |
| ISOLEUCINE CAMBRIDGE 80 MESH tit. 100% | 15 |
| VALINE CAMBRIDGE 40 MESH tit. 100% | 15 |
| POLYSORBATE MONTANOX 80 LIQUID FOOD | 10 |
| TOTAL | 100 |

TABLE d

| | % |
|---|---|
| Mixture D: Ingredient (Tit. + possible surd.) | weight |
| LEUCINE CAMBRIDGE 80 MESH tit. 100% | 72 |
| ISOLEUCINE CAMBRIDGE 80 MESH tit. 100% | 9 |
| VALINE CAMBRIDGE 40 MESH tit. 100% | 9 |
| POLYSORBATE MONTANOX 80 LIQUID FOOD | 10 |
| TOTAL | 100 |

Results

1. Mixtures of amino acids (tables a-d)

1.1. Mixtures of amino acids not treated according to the process: the amino acid powder (10 g) not treated with the process according to the present invention, once dispersed in 300 ml of water, without stirring, remains on the surface. After stirring for 1 minute, the powder still remains on the surface and agglomerates are formed. At the end of the stirring, after 3 minutes the powder visually still remains on the surface and agglomerated without dispersing or dissolving.

1.2 Mixtures of amino acids+6% Polysorbate: the powder (10 g), once dispersed in 300 ml of water, without stirring, already begins to disperse. After stirring, the powder visually appears already fully dispersed in the form of a suspension, after about 5 minutes from the end of the stirring, the powder is fully dissolved.

2. Protein (Whey protein isolate 90%)

2.1. Protein: the powder (30 g), once dispersed in 250 ml of water, without stirring, remains on the surface. After stirring for 1 minute, the powder still remains on the surface and agglomerates are formed. At the end of the stirring, after 3 minutes the powder visually still remains on the surface and agglomerated without dispersing or dissolving fully.

2.2 Protein+6% Polysorbate: the powder (30 g), once dispersed in 250 ml of water, without stirring, already begins to disperse. After stirring, the powder visually appears already fully dispersed in the form of a suspension, after about 3 minutes from the end of the stirring, the powder is fully dissolved.

3. Food hemp oil

3.1. Hemp oil (liquid)+FOS (fructo-oligosaccharides): the powder (5 g), once dispersed in 100 ml of water, fully dissolves in less than one minute and oil patches are observed on the surface due to the presence of hemp oil.

3.2 Hemp oil (liquid)+FOS+Polysorbate: the powder (5 g), once dispersed in 100 ml of water, is fully dissolved in less than one minute and no oil patches are observed on the surface due to the presence of hemp oil. This is because polysorbate emulsifies Hemp oil.

Furthermore, sequential application of hemp oil (liquid) and polysorbate 80 (liquid) on a soluble fibre such as FOS (solid), obtaining a solid powder formulated from these three components, 2 of which liquids, was particularly advantageous. This allows—with a single process—to obtain a powder soluble in water containing an oil (hemp oil in this case) which—once dissolved in water—does not have any oil patches on the surface thanks to polysorbate 80, which dispersed/emulsified the oil. Without polysorbate we observe the classic oil patches on the surface.

2. Comparison Processes (a-E)

Comparison Process (A): Dry Mixing in a Cube with Charging Agent.

Solid substrates in powder form with low or no wettability trend (in short, active) were dry mixed with charging agents.

Activities tested:
Palmitoylethanolamide PEA (<50 μm),
alpha-lactalbumin,
Boswellia (Boswellia serrata, titrated to 65% in boswellic acids).

Charging agents tested:
sugar (powder): extra white sugar (foodstuff) consisting of a single ingredient: colourless, pure and fluid sucrose crystals (Directive 2001/111/EC); polarisation 99.7° Z (ICUMSA method=International Commission for Uniform Methods of Sugar Analysis).
maltodextrin DE 19 (powder): CAS No. 9050-36-6, mixture of saccharides produced by means of controlled enzymatic hydrolysis of corn starch (foodstuff) followed by purification and freeze-drying;
characterisation (% w/w): loss on drying 5% max., protein content 0.15% max., sulphated ash 0.1% max., pH in solution 4.5-5.5; particle size: 5% max. (500 MIC.), about 65% (200 MIC.), 95% min. (40 MIC.).
D-mannitol (powder): CAS No. 69-65-8, characterisation (EP/USP/JP; % w/w): 97.0%-102.0%; melting point 165-170° C.; impurities: D-sorbitol 2.0% max., D-maltitol and isomalt 2.0% max., various 0.10% (total impurities: 2.0% max.); loss on drying 0.5% max.; average diameter>315 microns 10% max., >150 microns 45% min., >75 micron 90% max.

Said charging agents were used in high amounts (30% or 50% by weight with respect to the weight of the mixture comprising the active ingredient and the charging agent).

The purpose of said process (A) is to exploit the solubility of the charging agents to affect and increase the wettability of the active components in powdered form.

As per percentages reported in tables 1 to 3, the active components and the charging agents (total 1 kg) were mixed in a 2-litre cube for 10 minutes.

By testing the wettability of the solid formulations obtained (according to the process of paragraph 1.3 reported above), the result was negative given that the active components in powdered form remained on the water surface while the charging agents dissolved.

TABLE 1
Dry mixing in a cube (1 kg)

| | | % by weight Active | Visual degree of |
|---|---|---|---|
| Active | Charging | component/Charging | wettability of the |
| component | agent | agent | active component |
| PEA | Sugar | 70%/30% | Nil |
| PEA | Sugar | 50%/50% | Nil |
| Alpha-lactalbumin | Sugar | 70%/30% | Nil |
| Alpha-lactalbumin | Sugar | 50%/50% | Nil |
| Boswellia | Sugar | 70%/30% | Nil |
| Boswellia | Sugar | 50%/50% | Nil |

TABLE 2
Dry mixing in a cube (1 kg)

| | | % by weight Active | Visual degree of |
|---|---|---|---|
| Active | Charging | component/Charging | wettability of the |
| component | agent | agent | active component |
| PEA | Maltodextrin | 70%/30% | Nil |
| PEA | Maltodextrin | 50%/50% | Nil |
| Alpha-lactalbumin | Maltodextrin | 70%/30% | Nil |
| Alpha-lactalbumin | Maltodextrin | 50%/50% | Nil |
| Boswellia | Maltodextrin | 70%/30% | Nil |
| Boswellia | Maltodextrin | 50%/50% | Nil |

TABLE 3
Dry mixing in a cube (1 kg)

| | | % by weight Active | Visual degree of |
|---|---|---|---|
| Active | Charging | component/Charging | wettability of the |
| component | agent | agent | active component |
| PEA | Mannitol | 70%/30% | Nil |
| PEA | Mannitol | 50%/50% | Nil |
| Alpha-lactalbumin | Mannitol | 70%/30% | Nil |
| Alpha-lactalbumin | Mannitol | 50%/50% | Nil |
| Boswellia | Mannitol | 70%/30% | Nil |
| Boswellia | Mannitol | 50%/50% | Nil |

Comparison Process (B): Wet Granulation with Charging Agent.

Further tests were carried out by means of wet granulation—using the standard and known Kneader-Granulator-Dryer-Calibration sequence—of active components in the form of powders (PEA, alpha-lactalbumin or boswellia) with charging agents (sugar or maltodextrins or mannitol) in high amounts (30% and 50% by weight with respect to the weight of the mixture comprising the active ingredient and charging agent) (tables 4-6).

Wet granulation is a demanding process from process times and energy point of view, given that it involves the use of various machinery (kneader, granulator, dryer, calibration). In particular, the drying step is energy-consuming, given that it is necessary to evaporate the water up to a content in the final mixture below 5% by weight. After testing the wettability of the granulates obtained by said process (B), the result was unsatisfactory given that the active component in powdered form with little or no wettability remained on the water surface while the charging agents dissolved without significantly affecting the wettability of the powdered active components.

TABLE 4
Wet granulations (1.5 kg)

| | | % by weight Active | Visual degree of |
|---|---|---|---|
| Active | Charging | component/Charging | wettability of the |
| component | agent | agent | active component |
| PEA | Sugar | 70%/30% | Nil/low |
| PEA | Sugar | 50%/50% | Nil/low |
| Alpha-lactalbumin | Sugar | 70%/30% | Nil/low |
| Alpha-lactalbumin | Sugar | 50%/50% | Nil/low |
| Boswellia | Sugar | 70%/30% | Nil/low |
| Boswellia | Sugar | 50%/50% | Nil/low |

TABLE 5
Wet granulations (1.5 kg)

| | | % by weight Active | Visual degree of |
|---|---|---|---|
| Active | Charging | component/Charging | wettability of the |
| component | agent | agent | active component |
| PEA | Maltodextrin | 70%/30% | Nil/low |
| PEA | Maltodextrin | 50%/50% | Nil/low |
| Alpha-lactalbumin | Maltodextrin | 70%/30% | Nil/low |
| Alpha-lactalbumin | Maltodextrin | 50%/50% | Nil/low |
| Boswellia | Maltodextrin | 70%/30% | Nil/low |
| Boswellia | Maltodextrin | 50%/50% | Nil/low |

TABLE 6
Wet granulations (1.5 kg)

| | | % by weight Active | Visual degree of |
|---|---|---|---|
| Active | Charging | component/Charging | wettability of the |
| component | agent | agent | active component |
| PEA | Mannitol | 70%/30% | Nil/low |
| PEA | Mannitol | 50%/50% | Nil/low |
| Alpha-lactalbumin | Mannitol | 70%/30% | Nil/low |
| Alpha-lactalbumin | Mannitol | 50%/50% | Nil/low |
| Boswellia | Mannitol | 70%/30% | Nil/low |
| Boswellia | Mannitol | 50%/50% | Nil/low |

Comparison Process (C): Wet Granulation in a Fluid Bed Granulator with Charging Agents

Wet granulations were carried out in a fluid bed granulator using the same active components, charging agents and percentages of comparison processes (A) and (B) (table 7-9). Said method (C) provides for the atomisation of water on the mixture comprising the active ingredient and the charging agent inside the fluid bed to form a granulate. Using a fluid bed granulator, the times are faster with respect to the standard wet granulation. From an energy point of view, process (C) is demanding, given that it is necessary to evaporate water throughout the entire process by heating the process air in the fluid bed.

The wettability of the obtained solid formulations was low, although better than processes (A) and (B). It should be observed that the formulas with 50% of active component and 50% of charging agent gave a better wettability than the formulas with 70% of active component and 30% of charging agent.

TABLE 7
Wet granulation in a fluid bed granulator (1.5 kg)

| | | % by weight | | Visual |
|---|---|---|---|---|
| | | Active | | degree of |
| | | component/ | | wettability |
| Active | Charging | Charging | atomised | of the active |
| component | agent | agent | water (g) | component |
| PEA | Sugar | 70%/30% | about 350 g | Low |
| PEA | Sugar | 50%/50% | about 350 g | Low/Sufficient |
| Alpha-lactalbumin | Sugar | 70%/30% | about 300 g | Low |
| Alpha-lactalbumin | Sugar | 50%/50% | about 300 g | Low/Sufficient |
| Boswellia | Sugar | 70%/30% | about 400 g | Low |
| Boswellia | Sugar | 50%/50% | about 400 g | Low/Sufficient |

TABLE 8
Wet granulation in a fluid bed granulator (1.5 kg)

| | | % by weight Active | g | Visual degree of |
|---|---|---|---|---|
| Active | Charging | component/Charging | atomised | wettability of the |
| component | agent | agent | water | active component |
| PEA | Maltodextrin | 70%/30% | about 350 g | Low |
| PEA | Maltodextrin | 50%/50% | about 350 g | Low/Sufficient |
| Alpha-lactalbumin | Maltodextrin | 70%/30% | about 300 g | Low |
| Alpha-lactalbumin | Maltodextrin | 50%/50% | about 300 g | Low/Sufficient |
| Boswellia | Maltodextrin | 70%/30% | about 400 g | Low |
| Boswellia | Maltodextrin | 50%/50% | about 400 g | Low/Sufficient |

TABLE 9
Wet granulation in a fluid bed granulator

| | | % by weight Active | g | Visual degree of |
|---|---|---|---|---|
| Active | Charging | component/Charging | atomised | wettability of the |
| component | agent | agent | water | active component |
| PEA | Mannitol | 70%/30% | about 350 g | Low |
| PEA | Mannitol | 50%/50% | about 350 g | Low/Sufficient |
| Alpha-lactalbumin | Mannitol | 70%/30% | about 300 g | Low |
| Alpha-lactalbumin | Mannitol | 50%/50% | about 300 g | Low/Sufficient |
| Boswellia | Mannitol | 70%/30% | about 400 g | Low |
| Boswellia | Mannitol | 50%/50% | about 400 g | Low/Sufficient |

Comparison Process (D): Dry Granulations in the Presence of an Emulsifier and in the Absence of Charging Agents.

Wet granulations were carried out in a fluid bed granulator in the absence of charging agents using the same active components as in the comparison processes (A)-(C) and a wet agent (emulsifier), such as a liquid polysorbate 80 pure at 100% (Process D.a, table 10) or a sucrester (Process D.b, table 10).

(D.a) Process

Polysorbate 80 was added in a 10% percentage by weight with respect to the total weight of the mixture consisting of active component in powdered form and polysorbate. The resulting mixture was homogeneously mixed by means of a powder kneader. In said process (D) the water evaporation step is not present, therefore the process is more effective in terms of processing times and energy saving with respect to processes (A)-(C).

Basically, the polysorbate is added by means of a funnel to the powders and distributed by means of a mechanical mixing (for example, an impeller).

The wettability of the formulations obtained in said process (D) proved to be unsatisfactory, although said formulations have a partial wettability with respect to the almost zero wettability of the formulations obtained in processes (A)-(C).

(D.b) Sucrester

The sucrester in powdered form was added in a 10% percentage by weight with respect to the total weight of the mixture consisting of active component in powdered form and sucrester in powdered form. The resulting mixture was mixed in a standard manner in a cube. The result of the wettability of the formulations obtained in said process (D.b) was entirely unsatisfactory.

The sucrester used in the present experimental test is the sucrester commercially referred to as“Ryoto Sugar Ester P-1570”, an E-473 sucrester having the following characteristics (% by weight): mono- di- and tri-esters: not below 80%, free sugars: not above 4.0%, free fatty acids: not above 3.0% (estimated as oleic acid), sulfated ash: not above 1.5%, ethyl acetate and propan-2-ol and propylene glycol (single or combined): not more than 350 mg/kg, methanol: not more than 10 mg/kg, DMF: not more than 1 mg/kg, DMSO: not more than 2 mg/kg, 2-methyl-1-propanol: not more than 10 mg/kg, moisture: not more than 4.0%.

TABLE 10

| | | |
|---|---|---|
| Active component | Wettability agent | Visual degree of wettability |
| (90% w/w) | (10% w/w) | of the active component |

(D.a) Granulation in a powder kneader (1 kg)

| | | |
|---|---|---|
| PEA | Liquid polysorbate 80 pure | Low/Sufficient |
| Alpha-lactalbumin | Liquid polysorbate 80 pure | Low/Sufficient |
| Boswellia | Liquid polysorbate 80 pure | Low/Sufficient |

(D.b) Mixing in un cube (1 kg)

| | | |
|---|---|---|
| PEA | Powder sucrester | Nil |
| Alpha-lactalbumin | Powder sucrester | Nil |
| Boswellia | Powder sucrester | Nil |

Comparison Process (E): Wet Granulation in a Fluid Bed Granulator or in the Absence of Charging Agents

Further tests with polysorbate 80 were carried out using the fluid bed technique and diluting the polysorbate with water (10% weight/weight; 10 g of polysorbate/90 g of water) and applying the resulting polysorbate aqueous solution through said fluid bed on solid substrates in powdered form up to 10% weight/weight, as per table 11. The wettability of the solid formulations obtained in said process (E) were found to range between low and sufficient. However, said process (E) is inefficient and demanding in terms of processing time and energy, given that it is necessary to evaporate the water during processing.

TABLE 11
Wet granulation in a fluid bed granulator

| Active component | | Visual degree of wettability |
|---|---|---|
| 90% | Wetting agent 10% | of the active component |
| PEA | Aqueous solution | Low/Sufficient |
| Alpha-lactalbumin | of Polysorbate | Low/Sufficient |
| Boswellia | 80 at 10% w/w | Low/Sufficient |

1. A process for the preparation of a cold-liquid wettable solid formulation consisting of the steps of:
(I) providing a fluid bed granulator system comprising a process chamber, a process air distribution system, a means for atomising a wetting agent in liquid form, and
providing a peristaltic pump integrated or not integrated with said fluid bed system, wherein said atomisation means is positioned so as to be able to atomise the wetting agent in the process chamber;
(II) parallel or successively to step (I), providing—in a wetting agent container at room temperature—a wetting agent in liquid form, and connecting said wetting substance container to said peristaltic pump by means of connection means, wherein said wetting agent consists of a polysorbate and wherein said polysorbate does not comprise water;
(III) transferring—by means of a first transfer means—a solid substrate in powder form consisting of one or more hydrophobic compounds from a solid substrate container to said process chamber;
(VI) suspending—in a flow of said process air—said solid substrate in said process chamber by imparting a drive motion to said solid substrate;
(V) atomising—by means of said atomisation means—said wetting agent in the process chamber to obtain a cold-liquid wettable solid formulation, wherein said formulation comprises said solid substrate and said wetting agent and wherein said solid substrate consists of one or more hydrophobic compounds and said wetting agent consists of a polysorbate.

2. The process according to claim 1, wherein said process air imparts an elliptical or continuous circular crown motion to the solid substrate.

3. The process according to claim 1, wherein said atomisation means is a nozzle atomiser; preferably wherein said atomisation means atomises said wetting agent by means of an atomising compressed air at a pressure comprised in the range from 0.1·10⁵ Pa to 10·10⁵ Pa, preferably from 0.2·10⁵ Pa to 6·10⁵ Pa, more preferably from 0.3·10⁵ Pa to 4 bar 10⁵ Pa.

4. The process according to claim 1, wherein said polysorbate is selected from the group comprising or, alternatively, consisting of: polysorbate 20 such as polyoxyethylene sorbitan monolaurate, polysorbate 40 such as polyoxyethylene sorbitan monopalmitate, polysorbate 60 such as polyoxyethylene sorbitan monostearate, polysorbate 80 such as polyoxyethylene sorbitan monooleate, and a mixture thereof.

5. The process according to claim 1, wherein said solid formulation obtained from step (V) comprises said wetting agent in a percentage by weight comprised from 0.5% to 30% with respect to the weight of the solid formulation; preferably from 1% to 20%.

6. The process according to claim 1, wherein said solid formulation obtained from step (V) comprises said wetting agent in a percentage by weight comprised from 1% to 10% with respect to the weight of the solid formulation; preferably from 5% to 10%.

7. The process according to claim 1, wherein said one or more hydrophobic compounds are selected from the group comprising or, alternatively, consisting of: plant extracts, vitamins, minerals, amino acids, plant and animal solid fats, whey and casein proteins, polyphenols and mixtures thereof.

8. A cold-liquid wettable solid formulation obtained from the process according to claim 1, wherein said solid formulation consists of said solid substrate and said polysorbate, and wherein said solid substrate consists of said one or more hydrophobic compounds.

9. A composition comprising the solid formulation according to claim 8 and at least one acceptable pharmaceutical, food or cosmetic grade additive and/or excipient.

10. The composition according to claim 9, which is selected from dietary supplements, foods for special medical purposes (FSMPs), pharmaceutical compositions, medical device compositions and cosmetic compositions.

11. The composition according to claim 9, wherein said composition is in the form of powders or granules or tablets to be dissolved in water or in a water-based liquid or mouth dissolvable powders or granules.
